(19) United States
(12) Patent Application Publication
(10) Pub. No.: US 2013/0211059 A1
(43) Pub. Date: Aug. 15, 2013

(54) COMPOSITION SUPPRESSING MATRIX-METALLOPROTEINASE ACTIVITY

(71) Applicants: Toshihiro Tsuruda, Miyazaki (JP); Kazuo Kitamura, Miyazaki (JP)

(72) Inventors: Toshihiro Tsuruda, Miyazaki (JP); Kazuo Kitamura, Miyazaki (JP)

(73) Assignee: University of Miyazaki, Miyazaki (JP)

(21) Appl. No.: 13/818,126

(22) PCT Filed: Aug. 24, 2011
(86) PCT No.: PCT/JP2011/069677

§ 371 Date: May 2, 2013

(30) Foreign Application Priority Data
Aug. 24, 2010 (JP) 2010-187078

(51) Int. Cl. C07H 3/02

(52) U.S. Cl. CPC C07H 3/02
USPC 536/1.11

(57) ABSTRACT

An object of the present invention is to provide a composition having the effect of suppressing matrix metalloproteinase activity. Specifically, the present invention relates to a composition suppressing matrix metalloproteinase activity containing a glycolysis inhibitor as an active ingredient.

### TECHNICAL FIELD

The present invention relates to a composition having the effect of suppressing matrix metalloproteinase activity, for example.

### BACKGROUND ART

Abdominal aortic aneurysm (AAA) is a disease characterized by localized dilatation of abdominal aorta based upon atherosclerosis. AAA is predominantly observed in males 60 years of age or older and it occurs and develops asymptomatically, resulting in rupture. Hypertension and smoking are considered to be risk factors to promote aneurysm expansion; however, the detailed mechanisms of onset and development remain unknown. In the human AAA wall, thinning or disappearance of smooth muscle cells constituted tunica media layers has been thought to be a pathological feature. Hence, as human AAA develops, it becomes impossible to maintain the architecture of abdominal aorta due to the decrease of smooth muscle cells, and thus aneurysm gradually expands.

There is no effective pharmacotherapy against AAA. Thus, current medical intervention includes the strict blood pressure control, in parallel with the measurement of aneurysm size periodically using ultrasound or CT scan. Increasing size of AAA is considered to be an indication for surgery in order to prevent death from rupture. However, we have to concern with the age and his/her comorbidity leading to postoperative complications or lowered activity in daily life.

As described above, it is desired to develop a medical therapeutic method aiming at suppressing the expansion of aneurysm diameter.

Meanwhile, a glycolysis inhibitor is known to exert to suppress smooth muscle cell proliferation (Patent Document 1). For example, Patent Document 1 discloses that a glycolysis inhibitor such as 2-deoxyglucose (2-DG) is useful as a substance for curing wounds, including wounds associated with abnormal proliferation and/or migration of smooth muscle cells. However, Patent Document 1 does not disclose that a glycolysis inhibitor has the potential effect of suppressing the development of AAA.

### PRIOR ART DOCUMENT

### Patent Document

Patent Document 1 JP Patent Publication (Kohyo) No. 08-511041 A (1996)

### SUMMARY OF THE INVENTION

No effective pharmacotherapy has been conventionally available for AAA, as described above. Therefore, it is desired to develop a medical therapeutic method aiming at suppressing aneurysm diameter expansion.

In view of the above circumstances, an object of the present invention is to search for the underlying mechanism and to identify any factors responsible for suppressing AAA development. In addition, final goal is to provide a therapeutic agent having the effect of suppressing the development of a disease such as AAA.

As a result of intensive studies to achieve the above object, we found that glucose metabolism is enhanced in the human AAA wall, and that the glycolytic activity in the aneurysmal wall is associated with the protein expression of glucose transporters required for the incorporation of glucose into cells and a matrix-degrading enzyme, named matrix metalloproteinase (or also referred to as a matrix metalloprotease; hereinafter, referred to as “MMP”)-9 activity. Furthermore, we found that macrophages infiltrating the aneurysmal wall are the major cell source exhibiting the expression of glucose transporters.

It has been discovered that: MMP-9 activity was significantly suppressed in cultured macrophage cell line by the treatment with pharmacological inhibitors, capable of suppressing glucose metabolism or the expression and/or functions of a glucose transporter; administration of 2-DG significantly suppressed the aneurysm formation in mice model of aneurysm. Thus, the present invention has been completed.

The present invention encompasses the following (1) to (6).
(1) A composition suppressing MMP activity, containing a glycolysis inhibitor as an active ingredient.
(2) The composition suppressing MMP activity according to (1), wherein the glycolysis inhibitor is selected from the group consisting of 2-deoxyglucose, cytochalasin, and derivatives and salts thereof.
(3) The composition suppressing MMP activity according to (1) or (2), wherein the MMP is MMP in macrophages.
(4) The composition suppressing MMP activity according to any one of (1) to (3), wherein the MMP is MMP-9.
(5) A therapeutic agent for MMP-activation-related diseases, containing the composition suppressing MMP activity of any one of (1) to (4) as an active ingredient.
(6) The therapeutic agent for MMP-activation-related diseases according to (5), wherein the MMP-activation-related diseases are atherosclerosis or abdominal aortic aneurysm.

This description includes part or all of the contents as disclosed in the description and/or drawings of Japanese Patent Application No. 2010-187078, which is a priority document of the present application.

### BRIEF DESCRIPTION OF THE DRAWINGS

FIG. 1 illustrates (A) a graph showing the correlation between the expression of a glucose transporter protein (GULT-3) and MMP-9 activity in the human AAA wall and (B) photographs showing that immunoreactivity of GLUT-3 is localized in macrophages in the human AAA wall.

FIG. 2 illustrates graphs showing that MMP-9 activity was significantly decreased by the administration of (A) cytochalasin, (B) phloretin and (C) 2-DG in cultured macrophages, and of (D) 2-DG in ex vivo culture of the human AAA wall.

FIG. 3 shows (A) photographs and (B) a graph showing a result of administration of 2-DG to the calcium chloride application-induced aneurysm model in mice.

FIG. 4 illustrates a graph showing that administration of 2-DG suppressed aneurysm formation in the angiotensin II-induced apolipoprotein E knockout mice.

FIG. 5 illustrates graphs showing that 2-DG administration decreased gene expression levels associated with the development of atherosclerosis and aneurysm in cultured macrophages.

FIG. 6 illustrates graphs showing that 2-DG administration increased gene expression levels of SIRT1 in cultured macrophages.

### EMBODIMENTS FOR CARRYING OUT THE INVENTION

The present invention will be further described in detail. The composition suppressing MMP activity according to the present invention contains a glycolysis inhibitor as an active ingredient. For example, the composition suppressing MMP activity according to the present invention is administered to an animal such as a human, as a therapeutic agent for an MMP-activation-related disease to suppress MMP activity, so that the MMP-activation-related disease can be treated, prevented, or alleviated.

An example of MMP is MMP in macrophages. A specific example of MMP is MMP-9. Examples of other MMPs include MMP-1, MMP-2, and MMP-9 derived from components of vascular wall, such as endothelial cells, smooth muscle cells and fibroblasts.

Here, the term “glycolysis inhibitor” refers to a substance that inhibits glycolytic pathway for glycolysis. Examples of a glycolysis inhibitor include glucose transporter inhibitors such as 2-deoxyglucose, cytochalasin, phloretin, and derivatives and pharmacologically acceptable salts thereof. Glycolysis inhibitors may be commercially available products, or can be produced by conventional chemical synthesis methods and then used.

Also, the term “MMP activation” means that a part of the peptide of MMP proenzyme (referred to as “proenzyme” having no activity) is cleaved, and the activity (e.g., collagen degrading activity) is expressed.

The term “MMP-activation-related disease” refers to a disease, one of the causes of which is MMP activation, such as aortic aneurysm (e.g., atherosclerosis and AAA). Examples of other MMP-activation-related diseases include atherosclerosic plaque dissection, myocardial infarction, heart failure, restenosis, seizure, periodontal disease, tissue ulcer, wounds, dermatosis, cancer metastasis, vasculogenesis, age-related macular degeneration, fibrosis, chronic rheumatism, osteoarthritis, inflammatory disease due to migrating inflammatory cells, osteoarthritis, rheumatoid arthritis, septic arthritis, corneal ulcer, proteinuria, dystrophic epidermolysis bullosa, symptoms leading to inflammatory response, osteopenia due to MMP activity, temporomandibular arthrosis, nervous system demyelinating disease, regressive cartilage loss following tumor metastasis or traumatic arthropathy, coronary artery thrombosis derived from atherosclerotic plaque rupture, and birth control (JP Patent Nos. 3277170 and 3354941).

Moreover, the term “treatment of an MMP-activation-related disease” means that the symptoms of the MMP-activation-related disease is treated, prevented, or alleviated.

Examples of pharmaceutical ingredients that can be combined with a glycolysis inhibitor in the composition suppressing MMP activity according to the present invention include excipients, binders, disintegrators, surfactants, lubricants, fluid accelerators, flavoring agents, colorants, and aroma chemicals.

Examples of excipients include starch, lactose, saccharose, mannite, carboxy methylcellulose, corn starch, and inorganic salts.

Examples of binders include crystalline cellulose, crystalline cellulose carmellose sodium, methylcellulose, hydroxypropyl cellulose, low substituted hydroxypropyl cellulose, hydroxypropyl methylcellulose, hydroxypropyl methylcellulose phthalate, hydroxypropyl methylcellulose acetate succinate, carmellose sodium, ethyl cellulose, carboxy methyl ethyl cellulose, hydroxyethyl cellulose, wheat starch, rice starch, corn starch, potato starch, dextrin, gelatinized starch, partially gelatinized starch, hydroxypropyl starch, pullulan, polyvinylpyrrolidone, aminoalkyl methacrylate copolymer E, aminoalkyl methacrylate copolymer RS, methacrylate copolymer L, methacrylate copolymer, polyvinyl acetal diethyl aminoacetate, polyvinyl alcohol, gum Arabic, powdered acacia, agar, gelatin, white shellac, tragacanth, purified saccharose, and macrogol.

Examples of disintegrators include crystalline cellulose, methylcellulose, low substituted hydroxypropyl cellulose, carmellose, carmellose calcium, carmellose sodium, croscarmellose sodium, wheat starch, rice starch, corn starch, potato starch, partially gelatinized starch, hydroxypropyl starch, carboxymethyl starch sodium, and tragacanth.

Examples of surfactants include soybean lecithin, sucrose fatty acid ester, polyoxyl stearate, polyoxyethylene hydrogenated castor oil, polyoxyethylene polyoxypropylene glycol, sorbitan sesquioleate, sorbitan trioleate, sorbitan monostearate, sorbitan monopalmitate, sorbitan monolaurate, polysorbate, glyceryl monostearate, sodium lauryl sulfate, and lauromacrogol.

Examples of lubricants include wheat starch, rice starch, corn starch, stearic acid, calcium stearate, magnesium stearate, hydrous silicon dioxide, light anhydrous silicic acid, synthetic aluminum silicate, dried aluminum hydroxide gel, talc, magnesium alum inometasilicate, calcium hydrogen phosphate, anhydrous calcium hydrogen phosphate, sucrose fatty acid ester, waxes, hydrogenated plant oil, and polyethylene glycol.

Examples of fluid accelerators include hydrous silicon dioxide, light anhydrous silicic acid, dried aluminum hydroxide gel, synthetic aluminum silicate, and magnesium silicate.

Examples of dosage forms of the composition suppressing MMP activity according to the present invention include, but are not particularly limited to, oral preparations such as tablets, dust formulations, emulsions, capsules, granules, subtle granules, powders, liquid agents, syrups, suspensions, and elixir agents, or parenteral preparations such as injection preparations, drops, suppositories, inhalers, transdermal absorbents, transmucosal absorbents, adhesive preparations, sprays, and ointments.

Meanwhile, the content of the glycolysis inhibitor in the composition suppressing MMP activity according to the present invention can be adequately varied depending on purposes of administration, routes of administration, dosage forms, and the like. For example, the content is 0.01 mg or more and is preferably 0.1 mg or more.

The frequency of administration, dosage, and duration of administration for the composition suppressing MMP activity according to the present invention are not particularly limited and can be appropriately determined depending on patient age, gender, body weight, or the degree of severity of symptoms, route of administration, and the like. The frequency of administration ranges from once to three times a day and is preferably once a day, for example. The dosage of the active ingredient contained in the composition suppressing MMP activity according to the present invention may be 0.001 mg/kg body weight or more per day and preferably may be 0.01 mg/kg body weight or more per day, for example. Also, the duration of administration ranges from 1 to 7 days and preferably ranges from 1 to 2 days, for example.

The route of administration of the composition suppressing MMP activity according to the present invention can be appropriately determined depending on dosage forms and purposes for use. Examples thereof include peroral administration, parenteral administration (e.g., intrathecal administration, intraperitoneal administration, intravenous administration, intramuscular administration, subcutaneous administration, intrarectal administration, intranasal administration, and sublingual administration).

An example of a method for evaluating the MMP activity-suppressing effect of the composition suppressing MMP activity according to the present invention is a method that comprises culturing in vitro cells expressing MMP (e.g., macrophages expressing MMP-9) in the presence or the absence of the composition suppressing MMP activity according to the present invention, and then evaluating MMP activity in the cultured product using gelatin zymography. When MMP activity is significantly suppressed in cells cultured in the presence of the composition suppressing MMP activity according to the present invention, compared with cells (negative control) cultured in the absence of the composition suppressing MMP activity according to the present invention, it can be concluded that the composition suppressing MMP activity according to the present invention sufficiently suppresses MMP activity.

Meanwhile, examples of a method for pharmacologically evaluating the composition suppressing MMP activity according to the present invention as a therapeutic agent for an MMP-activation-related disease include a method using in vitro cells relating to an MMP-activation-related disease and a method using in vivo an MMP-activation-related disease model animal. For example, peri-aortic application of calcium chloride or an apolipoprotein E gene-modified mouse stimulated by angiotensin II can be used as an AAA animal model. Furthermore, apolipoprotein E knockout mice fed on high-fat diet or an atherosclerotic plaque destabilization model can also be used herein. The composition suppressing MMP activity according to the present invention is intraperitoneally administered to a mouse aneurysm model. Subsequently, when aneurysm formation is significantly suppressed compared with a mouse aneurysm model (negative control) to which the composition suppressing MMP activity according to the present invention has not been administered, it can be concluded that the composition suppressing MMP activity according to the present invention is effective for AAA. With the atherosclerosis model and the plaque destabilization model, therapeutic effects can be confirmed based on decreased atherosclerotic area as determined by oil red O staining, reduced thinning of plaque capsules as determined using tissue sections, and lowered MMP-9 activity.

Furthermore, in line with the composition suppressing MMP activity according to the present invention as described above, the present invention also relates to use of a glycolysis inhibitor in manufacture of a medicament for suppressing MMP activity in animals such as humans or other mammals, or a medicament for treating, preventing, or alleviating MMP-activation-related diseases. Here, the content of the glycolysis inhibitor in the medicament can be determined in line with the content of the glycolysis inhibitor in the composition suppressing MMP activity according to the present invention as described above.

Furthermore, the present invention relates to a method for suppressing MMP activity, comprising administering a glycolysis inhibitor in an effective dose to a patient (a human or an animal such as another mammal) requiring suppression of MMP activity, or a method for treating, preventing, or alleviating MMP-activation-related diseases, comprising administering a glycolysis inhibitor in an effective dose to a patient (a human or an animal such as another mammal) having an MMP-activation-related disease or a risk thereof. Here, the effective dose can be determined in line with the dosage of the glycolysis inhibitor contained in the composition suppressing MMP activity according to the present invention as described above.

The present invention will be more specifically described below by referring to Examples, but the technical scope of the present invention is not limited to these Examples.

### EXAMPLE 1

### Evaluation of the Expression of Glucose Transporter (GLUT-3) And the Matrix Metalloproteinase (MMP)-9 Activity In the Human AAA Wall

In this example, the expression of a GLUT-3 protein and the MMP-9 activity in the human AAA wall were evaluated.

The human AAA wall was homogenized, the obtained samples were evaluated for GLUT-3 protein expression by Western blot, and MMP-9 activity was determined by zymography. Western blot analysis was conducted by subjecting 10 μg of the extracted protein (quantified) to SDS-polyacrylamide gel electrophoresis for separation, subsequently transferring and immobilizing the resultant onto a membrane to prepare a blot, carrying out a reaction of the blot with an anti-GLUT-3 antibody for 1 hour and then with a secondary antibody, and then detecting signals. Meanwhile, zymogram analysis was conducted by subjecting 5 μg of the extracted protein (quantified) to electrophoresis on a zymogram gel plate to separate the protein. The method employed thereafter was carried out according to the instructions of a kit (Invitrogen). After 24 hours of a reaction at 37° C., zymogram gel was stained overnight with a Coomassie blue solution and then destained, and then enzyme activity was detected.

Furthermore, the GLUT-3 immunoreactive distribution in the human AAA wall was evaluated by an immunostaining method. In immunostaining, frozen sections of AAA were reacted with an anti-GLUT-3 antibody and an anti-CD68 antibody used as primary antibodies overnight at 4° C. Subsequently, the sections were incubated for 30 minutes with a secondary antibody labeled with fluorescein isothiocyanate, and with that labeled with Cy3, respectively. The results were observed with a confocal microscope (Olympus IX71).

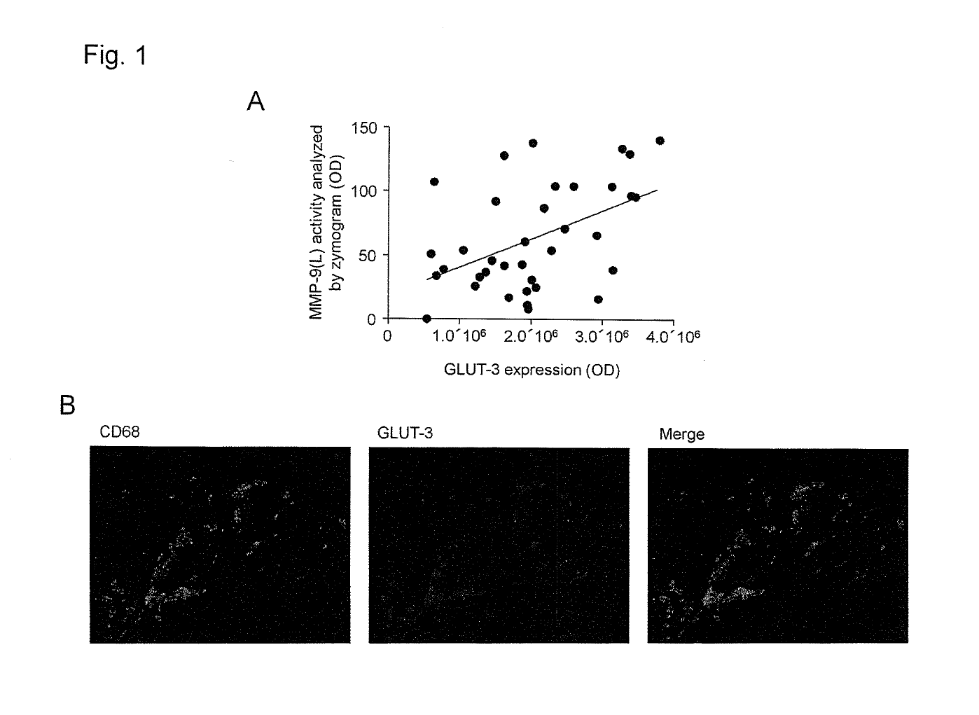

The results are shown in FIG. 1. Panel A is a graph showing MMP-9 activity (OD) using a zymogram on the vertical axis and GLUT-3 protein expression (OD) on the horizontal axis. In panel A, “MMP-9 (L)” denotes latent MMP-9 activity. As shown in the graph, a positive correlation (r=0.415, p=0.013) was observed between MMP-9 activity and GLUT-3 protein expression in the human AAA wall.

In the meantime, panel B illustrates photographs showing the immunostaining of the GLUT-3 protein in the human AAA wall. As shown in the photographs, the immunoreactivity (red) of GLUT-3 was co-localized (yellow (Merge)) with that of CD68-positive macrophages (green).

### EXAMPLE 2

### Evaluation of Suppression of MMP-9 Activity by the Administration of 2-DG, Cytochalasin, Or Phloretin In Cultured Macrophage Or Ex Vivo Culture of Human AAA

In this example, 2-DG, cytochalasin, or phloretin was administered to evaluate the suppression of MMP-9 activity in cultured macrophages or the human AAA wall, using a zymogram used in Example 1.

Monocytic cells (U937) were stimulated with 10 nmol/L phorbol ester (PMA) for differentiation to macrophages, and zymogram analysis revealed that MMP-9 activity was significantly enhanced in the macrophages.

In the meantime, U937 cells were pretreated with a glucose transporter inhibitor (cytochalasin or phloretin), and then stimulated with 10 nmol/L PMA. In addition, U937 cells were pretreated with 2-DG and then stimulated with 10 nmol/L PMA. Furthermore, 2-DG was administered to the ex vivo culture of human AAA wall.

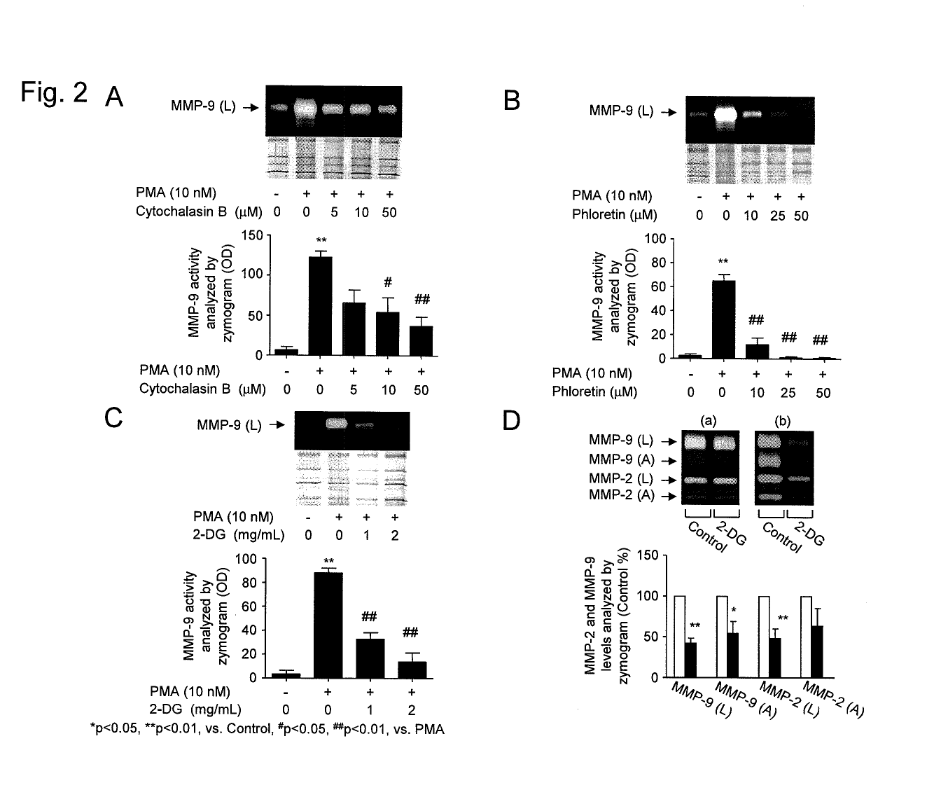

The results are shown in FIG. 2. Panels A to C show that effects of cytochalasin, phloretin, and 2-DG on the PMA-induced MMP-9 activity in cultured macrophages, respectively. Panel D shows that effects of 2-DG on MMP-9 activity in ex vivo culture of human AAA. In panels A to D, “MMP-9 (L)” denotes latent MMP-9 activity and “MMP-9 (A)” denotes active MMP-9 activity. In addition, in panel D, “MMP-2 (L)” denotes latent MMP-2 activity and “MMP-2 (A)” denotes active MMP-2 activity. Moreover, in panel D, “control” denotes an aqueous solvent not containing 2-DG.

As shown in FIG. 2, when U937 cells had been pretreated with a glucose transporter inhibitor (cytochalasin (A) or phloretin (B)), PMA-induced MMP-9 activity was decreased. Furthermore, when 2-DG had been administered to U937 cells to inhibit intracellular use of glucose, MMP-9 activity was decreased (C). 2-DG suppressed MMP-9 in ex vivo culture of the human AAA wall (D).

### EXAMPLE 3

### Evaluation of the Effect of 2-DG On Aneurysm Formation In A Mouse Model of Aneurysm

In this example, the effect of 2-DG was evaluated to suppress aneurysm formation in a mouse aneurysm model.

The mouse aneurysm model was induced by peri-aortic application of calcium chloride to 8-week-old male C57BL/6J mice.

2-DG was intraperitoneally administered at 100 mg/kg body weight or 1 g/kg body weight/day to the mouse aneurysm model for 28 days. After 28 days, the abdominal aorta was removed and then the diameter thereof was measured. An abdominal aorta was removed similarly from mice to which saline had been administered instead of 2-DG after application of calcium chloride as a negative control, and a mouse that had been treated in the same manner except for application of sodium chloride instead of calcium chloride as a control, and then the diameter of each thereof was measured.

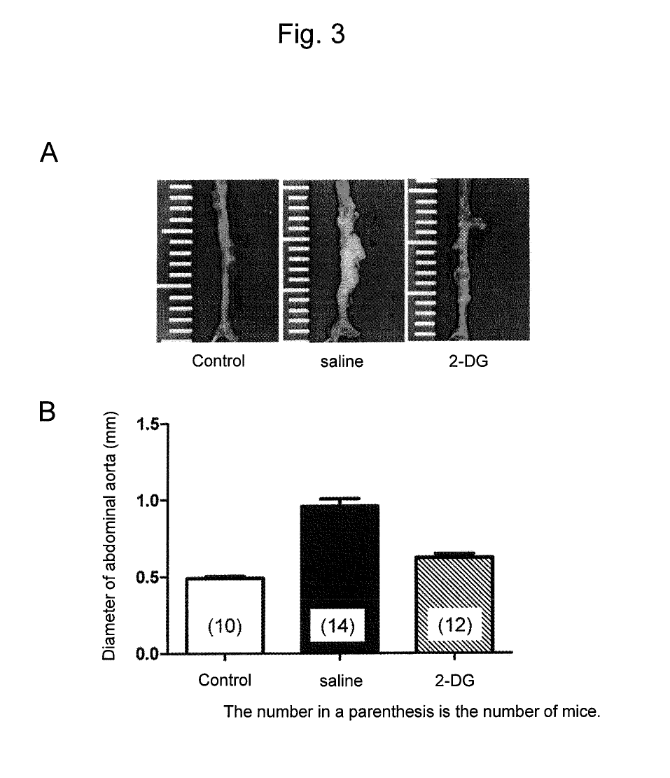

The results are shown in FIG. 3. Panel A shows typical photographs of the abdominal aorta removed from each group. Panel B is a graph showing the diameter (mm) of the abdominal aorta removed from each group.

As shown in FIG. 3, expansion of aortic diameter following the application of calcium chloride was suppressed by administration of 2-DG.

In addition, in this example, the tunica media layers, including smooth muscle, were damaged in the mouse aneurysm model group, whereas the mice treated with 2-DG maintained the vascular wall structure. Thus, the cytoprotective effect of 2-DG was confirmed.

Furthermore, the effect of administration of 2-DG was verified by another aneurysm model (that is, angiotensin II-induced apolipoprotein E knockout mice).

Each angiotensin II-induced apolipoprotein E knockout mouse was produced by subcutaneous administration of a pressor peptide, angiotensin II (1000 ng/kg/min), to a 12-week-old apolipoprotein E-deficient mouse via osmotic pump for 28 days.

After osmotic pump implantation, 2-DG was intraperitoneally administered at 1 g/kg body weight/day for 28 days (the number of mice (n)=14). After 28 days of administration, the abdominal aorta was removed and then the aortic diameter was measured. As negative control, the abdominal aorta was similarly removed from mice (the number of mice (n)=14) produced by administration of saline instead of 2-DG, and then the aortic diameter was measured.

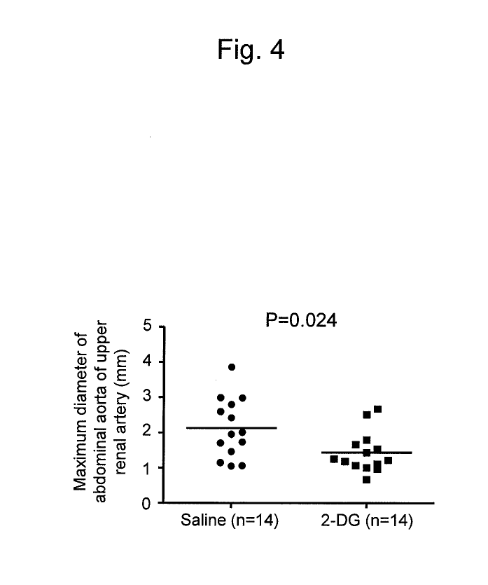

The diameter (mm) of the abdominal aorta removed from each group was shown in FIG. 4.

As shown in FIG. 4, also in this model, aneurysm formation was suppressed by administration of 2-DG, as revealed by measurement of aortic diameter.

### EXAMPLE 4

### Evaluation of the Effect of 2-DG On Gene Expressions Associated With Development of Atherosclerosis And/Or Aneurysm In Cultured Macrophages

In this example, the effects of 2-DG on gene expressions associated with development of atherosclerosis and/or aneurysm in cultured macrophages were evaluated by DNA microarray (Agilent). The evaluated genes associated with atherosclerosis induction- and/or aneurysm formation were MMP-1 and MMP-9, chemokines (CCL-2 and CCL-8) and inflammatory cytokines (TNF-α and IL-6) (Libby P., Inflammation in atherosclerosis, Nature 2002, 420 (6917): 868-874 and Tung W S, Lee J K, Thompson R W., Simultaneous analysis of 1176 gene products in normal human aorta and abdominal aortic aneurysms using a membrane-based complementary DNA expression array. J Vasc Surg. 2001, 34(1): 143-150).

Monocytic cells (U937) were stimulated with 10 nmol/L phorbol ester (PMA) for differentiation to macrophages. Some U937 cells were pretreated with 2-DG and then stimulated with 10 nmol/L PMA. As control cells, monocytic cells (U937) to which neither phorbol ester nor 2-DG had been administered were used. At 24 hours after stimulation, cells were collected, RNA was extracted, cDNA was constructed from RNA, and then the resultant was arrayed. The results of arraying were analyzed by GeneSpring GX10 software, so that the expression levels of the atherosclerosis induction- and/or aneurysm-related genes of each cell sample were evaluated.

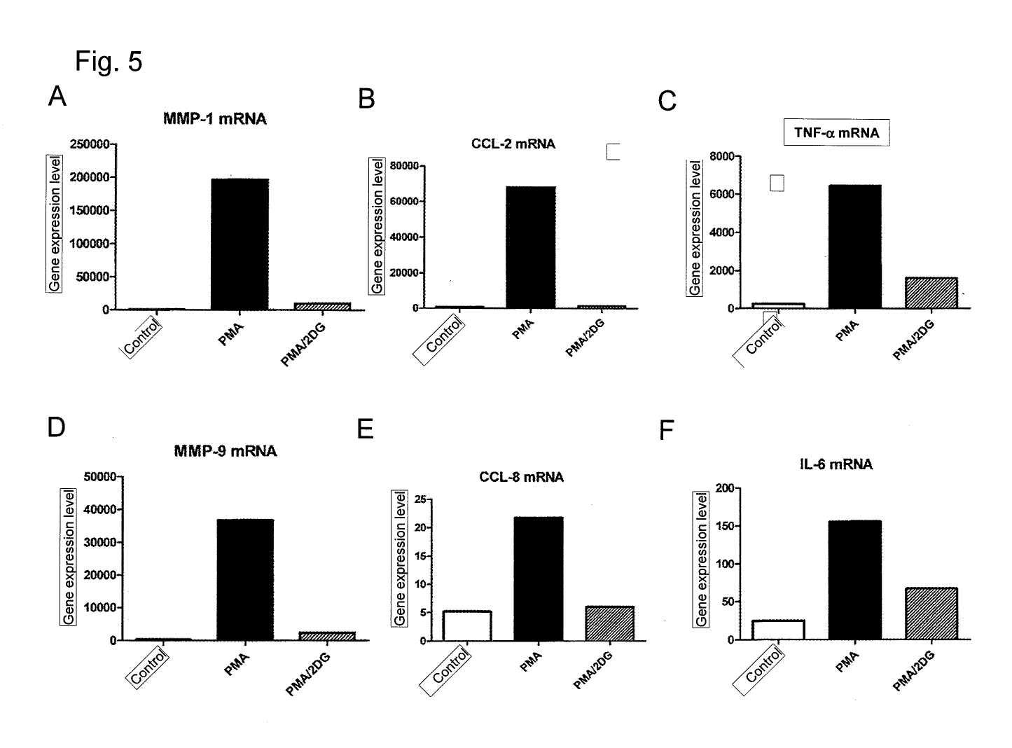

The results of the gene expression level of each of the atherosclerosis induction- and/or aneurysm-related genes in each cell sample are shown in FIG. 5. Panels A to F illustrate graphs showing the expression of MMP-1, CCL-2, TNF-α, MMP-9, CCL-8, and IL-6 at the mRNA level of each cell sample, respectively. Numerical figures on the vertical axis indicate the gene expression levels. The horizontal axis indicates each cell sample.

As shown in FIG. 5, phorbol ester (PMA) increased the expression level of each of the atherosclerosis induction- and/or aneurysm-related genes. In the meantime, the administration of 2-DG decreased the expression levels. Therefore, 2-DG is potentially an effective drug for suppressing atherosclerosis and/or aneurysm.

### EXAMPLE 5

### Evaluation of the Effect of 2-DG On SIRT1 Gene Expression In Cultured Macrophage

In this example, the effect of 2-DG was evaluated on the expression of SIRT1 gene in cultured macrophages. The SIRT1 gene is believed to possess a cytoprotection and longevity (Brachmann C B, Sherman J M, Devine S E, Cameron E E, Pillus L, Boeke J D. The SIR2 gene family, conserved from bacteria to humans, functions in silencing, cell cycle progression, and chromosome stability. Genes Dev. 1995; 9: 2888-2902). Gene expression was evaluated at the mRNA level in a manner similar to that used in Example 4.

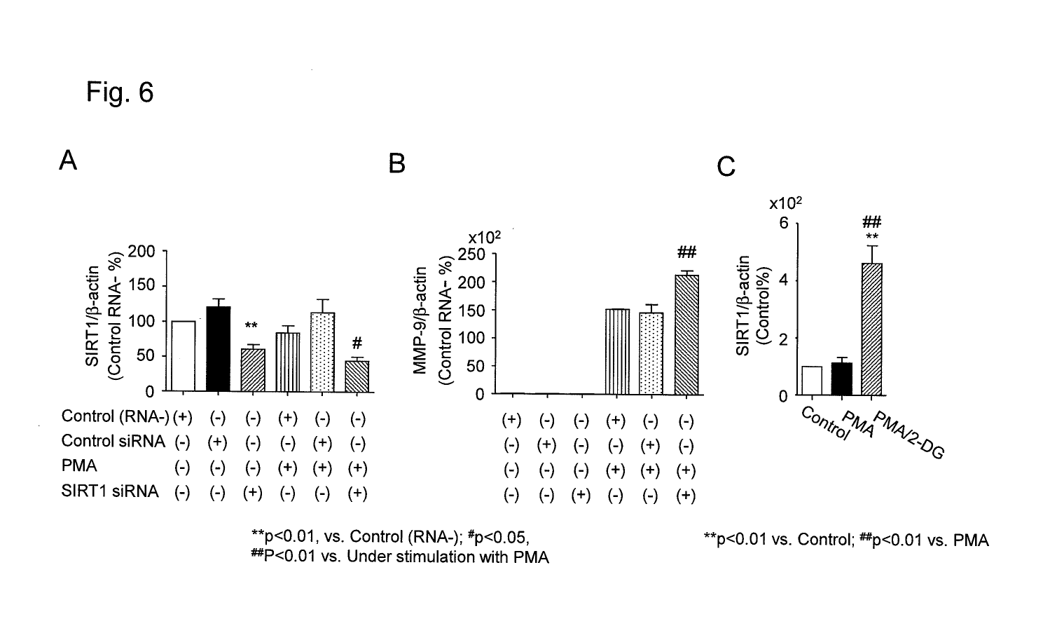

It was confirmed that when the expression of the SIRT1 gene of monocytic cells (U937) had been suppressed by an RNA interference method, the expression of the SIRT1 gene was suppressed under control conditions. It was also confirmed that when monocytic cells (U937) had been stimulated with 10 nmol/L phorbol ester (PMA) for differentiation to macrophages, the expression of the SIRT1 gene was suppressed by the RNA interference method (panel A in FIG. 6). In panels A and B of FIG. 6, “control (RNA-)” denotes the results for a sample containing no siRNA, to which only a solvent was added; “control siRNA” denotes scramble siRNA (having the same nucleotide proportions as those of siRNA (SIRT1 siRNA) for suppressing target RNA (SIRT1 RNA) and comprising a gene sequence differing from those of any genes).

Panel B in FIG. 6 shows the expression of the MMP-9 gene in monocytic cells induced by PMA under the same condition. This result suggests that when the expression of the SIRT1 gene is suppressed, the expression of the MMP-9 gene is significantly increased.

In the meantime, 2-DG (2 mg/mL) was pretreated to the U937 cell culture solution, and stimulated them with PMA. As control cells, monocytic cells (U937) to which neither phorbol ester nor 2-DG had been administered and monocytic cells (U937) to which only phorbol ester had been administered were used. At 24 hours after stimulation, SIRT1 gene expression levels were compared, and the results are shown in panel C of FIG. 6.

As shown in panel C of FIG. 6, the administration of 2-DG resulted in a significantly increased SIRT1 gene expression level.

Taken together, 2-DG suppresses MMP-9 activity in macrophages, in part through the induction of the SIRT1 gene.

### INDUSTRIAL APPLICABILITY

According to the present invention, a composition that can suppress MMP activity and is effective for treating, alleviating, and preventing MMP-activation-related diseases can be provided.

All publications, patents, and patent applications cited in this description are herein incorporated by reference in their entirety.

1. A composition suppressing matrix metalloproteinase activity, containing, as an active ingredient, a glycolysis inhibitor selected from the group consisting of 2-deoxyglucose, a derivative and a salt thereof.

2. (canceled)

3. The composition suppressing matrix metalloproteinase activity of claim 1, wherein the matrix metalloproteinase is a matrix metalloproteinase in macrophages.

4. The composition suppressing matrix metalloproteinase activity of claim 1, wherein the matrix metalloproteinase is matrix metalloproteinase-9.

5. A therapeutic agent for matrix metalloproteinase-activation-related diseases containing, as an active ingredient, the composition suppressing matrix metalloproteinase activity of claim 1.

6. The therapeutic agent for matrix metalloproteinase-activation-related diseases of claim 5, wherein the matrix metalloproteinase-activation-related diseases are atherosclerosis or abdominal aortic aneurysm.

7. A composition regulating expression of an atherosclerosis-related or an abdominal aortic aneurysm-related gene containing, as an active ingredient, a glycolysis inhibitor selected from the group consisting of 2-deoxyglucose, a derivative and a salt thereof.

8. The composition regulating expression of the atherosclerosis-related or the abdominal aortic aneurysm-related gene of claim 7, wherein the atherosclerosis-related or the abdominal aortic aneurysm-related gene is selected from the group consisting of a gene encoding a chemokine, a gene encoding an inflammatory cytokine and an SIRT-1 gene.

9. The composition regulating expression of the atherosclerosis-related or the abdominal aortic aneurysm-related gene of claim 8, wherein the chemokine is selected from the group consisting of CCL-2 and CCL-8.

10. The composition regulating expression of the atherosclerosis-related or the abdominal aortic aneurysm-related gene of claim 8, wherein the inflammatory cytokine is selected from the group consisting of TNF-α and IL-6.

11. A therapeutic agent for an atherosclerosis-related or an abdominal aortic aneurysm containing, as an active ingredient, the composition regulating expression of the atherosclerosis-related or the abdominal aortic aneurysm-related gene of claim 7.

12. A therapeutic agent for an atherosclerosis-related or an abdominal aortic aneurysm containing, as an active ingredient, the composition suppressing matrix metalloproteinase activity of claim 1 and the composition regulating expression of an atherosclerosis-related or an abdominal aortic aneurysm-related gene containing, as an active ingredient, a glycolysis inhibitor selected from the group consisting of 2-deoxyglucose, a derivative and a salt thereof.

13. The composition suppressing matrix metalloproteinase activity of claim 3, wherein the matrix metalloproteinase is matrix metalloproteinase-9.

14. A therapeutic agent for matrix metalloproteinase-activation-related diseases containing, as an active ingredient, the composition suppressing matrix metalloproteinase activity of claim 3.

15. A therapeutic agent for matrix metalloproteinase-activation-related diseases, containing, as an active ingredient, the composition suppressing matrix metalloproteinase activity of claim 4.

16. A therapeutic agent for an atherosclerosis-related or an abdominal aortic aneurysm containing, as an active ingredient, the composition regulating expression of the atherosclerosis-related or the abdominal aortic aneurysm-related gene of claim 8.

17. A therapeutic agent for an atherosclerosis-related or an abdominal aortic aneurysm containing, as an active ingredient, the composition regulating expression of the atherosclerosis-related or the abdominal aortic aneurysm-related gene of claim 9.

18. A therapeutic agent for an atherosclerosis-related or an abdominal aortic aneurysm containing, as an active ingredient, the composition regulating expression of the atherosclerosis-related or the abdominal aortic aneurysm-related gene of claim 10.

19. A therapeutic agent for an atherosclerosis-related or an abdominal aortic aneurysm containing, as an active ingredient, the composition suppressing matrix metalloproteinase activity of claim 3 and the composition regulating expression of an atherosclerosis-related or an abdominal aortic aneurysm-related gene containing, as an active ingredient, a glycolysis inhibitor selected from the group consisting of 2-deoxyglucose, a derivative and a salt thereof.

20. A therapeutic agent for an atherosclerosis-related or an abdominal aortic aneurysm containing, as an active ingredient, the composition suppressing matrix metalloproteinase activity of claim 4 and the composition regulating expression of an atherosclerosis-related or an abdominal aortic aneurysm-related gene containing, as an active ingredient, a glycolysis inhibitor selected from the group consisting of 2-deoxyglucose, a derivative and a salt thereof.
